(19) United States
(12) Patent Application Publication
(10) Pub. No.: US 2025/0011726 A1
(43) Pub. Date: Jan. 9, 2025

(54) METHODS AND COMPOSITIONS FOR MAINTAINING AGGREGATE STABILITY AND PLURIPOTENCY OF STEM CELLS

(71) Applicant: Trailhead Biosystems Inc., Beachwood, OH (US)

(72) Inventors: Michael BUKYS, Lakewood, OH (US); Haneen YEHYA, Parma, OH (US)

(21) Appl. No.: 18/763,151

(22) Filed: Jul. 3, 2024

Related U.S. Application Data
(60) Provisional application No. 63/572,658, filed on Apr. 1, 2024.
(60) Provisional application No. 63/525,083, filed on Jul. 5, 2023.

(51) Int. Cl. C12N 5/074

(52) U.S. Cl. CPC C12N 5/0696; C12N 2500/50; C12N 2500/60

(57) ABSTRACT

Methods for maintaining aggregate stability and pluripotency of human stem cells are provided using chemically-defined culture media that includes heparin sodium salt and polyethylene glycol. Methods of modulating aggregate size and/or stability using chemically-defined culture media are also provided. The methods can be used with, for example, induced pluripotent stem cells or embryonic stem cells. Culture media and kits are also provided.

### RELATED APPLICATION

This application claims priority to U.S. Provisional Application No. 63/525,083, filed Jul. 5, 2023, and U.S. Provisional Application No. 63/572,658. filed April 1. 2024, the entire contents of each of which is hereby incorporated by reference.

### BACKGROUND OF THE INVENTION

Stem cell-derived cell therapies hold the potential for treatment of regenerative clinical indications. Human pluripotent stem cells (hPSCs) can proliferate continuously and have the capacity to differentiate into any human cell type. For growth of pluripotent stem cells, conventional static culture flasks are impractical for large scale production needs as they require frequent manual intervention. Static culture has a limited ability to scale up thus restricting its use. Suspension culturing can be used to produce target cells in large quantities. Adherent cultures have a limited ability to scale up and result in high batch-to-batch variability and a lack of cost effectiveness. Continuously stirred-tank suspension bioreactors provide a culture that generates aggregates that are more biologically comparable to an in vivo environment (Nogueira et al. (2019) J. Biol. Eng. 13:74). In addition, bioreactors can provide continuous monitoring of environmental factors, including temperature, pH, oxygen, and nutrients in a sterile environment required to produce clinical grade cells. Maintaining the pluripotent state in a large-scale suspension environment will improve reproducibility and cell quality, but it requires control over media composition, aggregation and physicochemical stresses exerted on cells.

hPSC expansion in bioreactors presents several challenges including cell clumping, shear stress, complex media composition and cost. Cell clumping or aggregate fusion can cause heterogenous cell populations to arise (Otsuji et al. (2014) Stem Cell Reports 2:746). Shear stress leads to aggregate breakage, DNA-breaks, resulting in cell death and karyotypic instability (Abbasalizadeh et al. (2012) Tissue Eng. Part C, Methods 18:831-851). An environment that can prevent unwanted cell adhesion and maintain aggregate stability while maintaining pluripotency for manufacturing expansion is in demand. Cell State control is particularly relevant, as heterogeneous populations presents a serious risk, as incomplete differentiation impacts both clinical safety and potency (Otsuji et al. (2014) Stem Cell Reports 2:746). While simple elements such as temperature and pH can be controlled and monitored, molecular component interactions, concentration sensitivity, chemical stability of additives, when combined with physical stresses, mechanical forces present all combine into a very challenging production problem.

Certain process parameters that affect hPSC culture performance have been described in the art (see e.g., Liu et al. (2013) World J. Stem Cells 5:124-135; Teramura et al. (2012). Biochem. Biophys. Res. Comm. 417:836-841; Saha et al. (2008) Biophys. J. 94:4123-4133; Kim et al. (2017) Stem Cell. Res. Therap. 8:139; Goetzke et al. (2018) Cell. Mol. Life Sci. 75:3297-3312). While some progress has been made, there remains a need for effective methods and compositions for modulating aggregation of pluripotent stem cells in culture, particularly approaches that are suitable for large scale bioreactor culture.

### SUMMARY OF THE INVENTION

This disclosure provides methods and culture compositions for maintaining and/or modulating aggregate stability in hPSC cultures, such as in a bioreactor. Utilizing a design of experiments (DoE) approach, media additives were evaluated that have versatile properties, such as reducing shear stress by decreasing surface tension, enhancing extracellular matrix and cell membrane interaction, increasing aggregate stability, and preventing aggregate fusion. Multiple response parameters were chosen to assess cell growth, pluripotency maintenance and aggregate stability in response to five additive inputs, and mathematical models were generated and tuned for maximal predictive power. The methods and compositions of the disclosure are based, at least in part, on the discovery that use of a culture media comprising the combination of heparin sodium salt (HS) and polyethylene glycol (PEG) for hPSC culture maintains aggregate stability, as well as pluripotency, exceptionally well as compared to other agents alone or in combination.

The methods and compositions provide the ability to remove unwanted aggregate growth during the differentiation process of hPSCs. Moreover, they allow for control of aggregate size and can be adapted to bioreactor culture.

The methods and compositions herein enable better control over aggregate formation and growth within suspension cultures of hPSCs maintained within bioreactors. This disclosure provides methods that can 1) limit the ability of aggregates to clump and stick together, thereby allowing aggregates to grow in size only through cellular expansion and 2) enable aggregates to fuse together enabling stabile growth of aggregates grown in suspension or within bioreactors. The media additives described herein can be used in conjunction with any basal media to regulate aggregate growth within a suspension culture. This allows for the continued growth of aggregates without the disruption of the aggregate structure. Furthermore, this allows for both growth within the pluripotent state as well as better differentiation efficacy.

Accordingly, in one aspect, the disclosure pertains to a method of maintaining aggregate stability of human pluripotent stem cells (hPSCs), the method comprising culturing the human pluripotent stem cells in a culture media comprising heparin sodium salt (HS) and polyethylene glycol (PEG). In an embodiment, the hPSCs are human induced pluripotent stem cells (hiPSCs). In an embodiment, the hPSCs are human embryonic stem cells. In an embodiment, aggregation of the hPSCs is inhibited compared to culture in a media lacking HS and PEG. In an embodiment, pluripotency of the hPSCs is maintained in culture (e.g., as evidenced by expression of one or more stem cell markers).

In an embodiment, heparin sodium salt is present in the culture media at a concentration within a range of 0.05-0.2 ug/ml. In an embodiment, HS is present in the culture media at a concentration of 0.1 ug/ml.

In an embodiment, PEG is present in the culture media at a concentration within a range of 5-20 mg/ml. In an embodiment, PEG is present in the culture media at a concentration of 10 mg/ml.

In an embodiment, the culture media further comprises polyvinyl alcohol (PVA). In an embodiment, PVA is present in the culture media at a concentration within a range of 0.5-2 mg/ml. In an embodiment, PVA is present in the culture media at a concentration of 1 mg/ml.

In an embodiment, the culture media further comprises dextran sulfate (DS). In an embodiment, DS is present in the culture media at a concentration within a range of 50-150 ug/ml. In an embodiment, DS is present in the culture media at a concentration of 100 ug/ml.

In an embodiment, the culture media further comprises Pluronic acid F68 (PA). In an embodiment, PA is present in the culture media at a concentration within a range of 0.5-2 mg/ml. In an embodiment, PA is present in the culture media at a concentration of 1 mg/ml.

In an embodiment, the culture media comprises a basal media selected from the group consisting of Essential 8, DMEM, IMDM and RPMI. In an embodiment, the basal media is Essential 8.

In an embodiment, culturing of the hPSCs in a culture media comprising HS and PEG increases expression of at least one pluripotent stem cell marker on the hPSCs. In an embodiment, the pluripotent stem cell marker is selected from the group consisting of SOX2, OCT4, NANOG, TRA-1-60 and SSEA4. In an embodiment, the pluripotent stem cell marker is SOX2.

In an embodiment, culturing the hPSCs in a culture media comprising HS and PEG, following by challenging for ecto-, endo-or meso-dermal activity, increases expression of one or more ecto-, endo-or meso-dermal markers, respectively. In an embodiment, ectodermal markers are PAX6 and Nestin. In an embodiment, endodermal markers are FOXA2 and SOX17. In an embodiment, mesodermal markers are TNNT2 and Brachyury (T).

In an embodiment, culture of the hPSCs in a culture media comprising HS and PEG leads to downregulation of expression of certain genes. In an embodiment, expression of TGFβ family genes, such as BMP2 and/or BMP4, is downregulated. In an embodiment, expression of Wnt family genes, such as Wnt4, FRZB, FZD5 and/or FRZ8, are downregulated. In an embodiment, expression of retinoic acid family genes, such as CYP26A1, CYP26C1, DHRS3 and/or CRABP2, are downregulated. In an embodiment, ectodermal genes, such as SOX1, OLIG3, LHX5 and/or OTX2, are downregulated.

In an embodiment, the disclosure provides a method of maintaining aggregate stability of human pluripotent stem cells (hPSCs), the method comprising culturing the human pluripotent stem cells in a culture media comprising 0.1 ug/ml heparin sodium salt (HS), 10 mg/ml polyethylene glycol (PEG), 1 mg/ml polyvinyl alcohol (PVA) and 100 ug/ml dextran sulfate (DS).

In another aspect, the disclosure pertains to an aqueous culture media for maintaining aggregate stability and pluripotency of human pluripotent stem cells (hPSCs) comprising heparin sodium salt (HS) and polyethylene glycol (PEG). In an embodiment, HS is present at a concentration within a range of 0.05-0.2 ug/ml, e.g., at a concentration of 0.1 ug/ml. In an embodiment, PEG is present at a concentration within a range of 5-20 mg/ml, e.g., at a concentration of 10 mg/ml. In an embodiment, the media further comprises polyvinyl alcohol (PVA), such as within a concentration range or at a concentration disclosed herein. In an embodiment, the media further comprises dextran sulfate (DS), such as within a concentration range or at a concentration disclosed herein. In an embodiment, the media further comprises Pluronic acid F68 (PA), such as within a concentration range or at a concentration disclosed herein.

In another aspect, the disclosure pertains to an isolated cell culture of human pluripotent stem cells, the culture comprising human pluripotent stem cells (hPSCs) cultured in a culture media comprising heparin sodium salt (HS) and polyethylene glycol (PEG). In an embodiment, the hPSCs are human induced pluripotent stem cells (hiPSCs). In an embodiment, the hPSCs are human embryonic stem cells. In an embodiment, the culture media further comprises polyvinyl alcohol (PVA). In an embodiment, the culture media further comprises dextran sulfate (DS). In an embodiment, the culture media further comprises Pluronic acid F68 (PA). Representative concentration ranges and concentrations for the media components are disclosed herein.

In another aspect, the disclosure pertains to a method of modulating size and/or stability of human pluripotent stem cell aggregates in culture using the chemically-defined culture media disclosed herein. In embodiments, the method comprises:

culturing human pluripotent stem cells (hPSCs) in a culture media comprising heparin sodium salt (HS) and polyethylene glycol (PEG), wherein HS and PEG are at a concentration and molecular weight sufficient to modulate the size or stability of hPSCs aggregates in culture.

In embodiments, the concentration and/or molecular weight of PEG is selected to destabilize larger aggregates. In embodiments, the concentration and/or molecular weight of PEG is selected to mediate creation of smaller aggregates from larger aggregates. In embodiments, the concentration and/or molecular weight of PEG is selected to initiate formation of daughter aggregates as a function of aggregate size or growth.

In embodiments, the concentration of PEG in the culture media is varied to select a concentration sufficient to modulate the size or stability of the hPSCs aggregates in culture. In other embodiments, the molecular weight of PEG in the culture media is varied to select a molecular weight sufficient to modulate the size or stability of the hPSCs aggregates in culture. In yet other embodiments, both the concentration and the molecular weight of PEG in the culture media are varied to select a concentration and molecular weight sufficient to modulate the size or stability of the hPSCs aggregates in culture.

In embodiments, the concentration of PEG in the culture media is 0.5%. In embodiments, the concentration of PEG in the culture media is 1%. In embodiments, the concentration of PEG in the culture media is 2%. In embodiments, the molecular weight of PEG in the culture media is 1500 Daltons. In embodiments, the molecular weight of PEG in the culture media is 8000 Daltons. In addition to HS and PEG, the culture media can comprise other media components as described herein.

Other features and advantages of the invention will be apparent from the following detailed description and claims.

### BRIEF DESCRIPTION OF THE DRAWINGS

FIG. 1 shows results from evaluation of human pluripotent cell medium additives with a bioreactor expansion assay. Results are normalized to 3 replicate center point experiments that have all components at mid-concentration levels. Optimization are completed using CR-0001 hiPSC line with a short 5-day aggregate growth experiment. The graph shows the coefficient plot of doubling time based on the response results of all variables and interactions detected.

FIGS. 2A-2C show the results for cell growth optimizer experiments. FIG. 2A shows the Dynamic Profile plot (PLS) for optimizer set point. FIG. 2B shows the optimizer results of doubling time parameter. FIG. 2C shows the validation testing for the doubling time optimizer in comparison to original media.

FIGS. 3A-3C show the results for pluripotency marker optimization experiments. FIG. 3A shows MODDE model optimizers for pluripotency markers SOX2, OCT4 and NANOG. FIG. 3B shows the data coefficients of each pluripotency marker. FIG. 3C shows the pluripotency Validation Optimizer in comparison to original media.

FIGS. 4A-4C show results for aggregate stability optimizer experiments. FIG. 4A is a schematic showing expected aggregate growth in different scenarios. FIG. 4B shows Aggregate % Error optimizer results for maximization, predicted target and minimization of error. FIG. 4C shows aggregate stability optimizer validation in comparison to original media.

FIGS. 5A-5C show results for aggregate stability optimizer results. FIG. 5A shows results of a MODDE optimizer for pluripotency marker OCT4, doubling time and aggregate stability. FIG. 5B shows validation results of the optimizer when testing for pluripotency markers on Flow. FIG. 5C shows validation results of the optimizer when testing for growth rate.

FIG. 6 shows RNA-seq results presented as a volcano plot showing the comparison of the pluripotent stem cell medium disclosed herein to the control medium (E8).

FIG. 7A shows RNA-seq results presented as a heatmap comparison of selected genes evaluating pluripotency, differentiation and other parameters.

FIG. 7B shows RNA-seq results presented as a heatmap comparison of the top differentially expressed genes between the pluripotent stem cell medium (E8+PEG+HS) and the control medium (E8).

FIGS. 8A-8E show results demonstrating that aggregate size can be defined as a function of both the MW and the concentration of PEG used. FIG. 8A shows representative images demonstrating aggregates produced in the one condition and how they are being measured using ImageJ. FIG. 8B shows parameters measured for each of the six conditions tested. FIG. 8C shows Scatter dot plot showing the average and distribution of other sized aggregates in the six different conditions. FIG. 8D is a graph showing the relationship between the concentration of PEG used and the overall distribution in aggregate size. FIG. 8E are graphs highlighting the decreasing variance of aggregate size as a function of the concentration of PEG.

FIGS. 9A-9C show results demonstrating that aggregate size is not regulated through contact inhibition. FIG. 9A shows RNA sequencing-based assessment of gene known to be down-regulated during contact inhibition. FIG. 9B shows transcript-based assessment of some common proliferation markers. FIG. 9C shows transcript-based assessment of some genes associated with different phases of the cell cycle.

FIGS. 10A-10B shows results relating to digestion-free serial passaging of iPSC aggregates. FIG. 10A is a schematic showing the serial passaging scenario of iPSC aggregates grown in the presence of PEG/HS. FIG. 10B shows growth curves for 2-iPSC cell lines that were serial passaged using this method.

FIGS. 11A-11F illustrate a proposed mechanism of continued aggregate growth, with a schematic showing aggregate division A′-E′ and images showing the process of aggregate division.

### DETAILED DESCRIPTION OF THE INVENTION

Described herein are methodologies and compositions that allow for modulating aggregation of human pluripotent stem cells (hPSCs) in culture under chemically-defined culture conditions using a small molecule based approach. As described in Example 2, a High-Dimensional Design of Experiments (HD-DoE) approach was used to simultaneously test multiple process inputs (e.g., small molecules). These experiments allowed for the identification of chemically-defined culture media that in particular allow for modulation of cell aggregation. The disclosure provides defined combinatorial media formulations that directly influence pluripotency, growth and aggregate stability of hPSC cultures that were identified using a five-dimensional DoE based approach. As described herein, the two main components that positively influenced the majority of the identified critical attributes were heparin sodium salt (HS) and polyethylene glycol (PEG). Interactions between heparin sodium salt and PEG were well modeled, predicted, and enabled a better understanding of controlling aggregate stability.

While together improving iPSC aggregate culturing, PEG and heparin sodium salt have different effects on cellular aggregates. PEG is known to induce cell aggregation and promote the fusion of larger cell aggregates at higher concentrations (10% and above), while also capable of inhibiting aggregation in other applications at lower concentrations (Castellanos et al. (2003) J. Controlled Release 88:135-145). The latter capability is similar to the expected effects of heparin sodium salt, as heparin sodium salt is a known anti-coagulant for cellular aggregation, preventing fusion. Being able to detect this synergy within compounds that have an overall opposing effect on the culture reinforces the strength of the Design of Experiments approached used in this disclosure. The only compounds used in the examples that did not increase ‘aggregate % error’ were PEG and heparin sodium salt. Modelling PEG indicated the overall effect was a reduced standard deviation of the aggregate diameter size, though no effect on doubling time was observed. The aggregate deviation and diameter growth rate increased slightly in the presence of PEG. While the addition of heparin sodium salt did not show a strong impact on aggregate size or growth rate individually it was shown to have strong positive interaction in the presence of PEG. Accordingly, the disclosure provides compositions and methods utilizing the unique combination of heparin sodium salt and PEG in culture of hPSCs to thereby modulate, and in particular inhibit, cell aggregation.

Moreover, as demonstrated in Example 5, varying the concentration and/or molecular weight of PEG used in the culture modulates the size and/or stability of hPSC aggregates in culture. Accordingly, the disclosure also provides methods of modulating the size and/or stability of hPSC aggregates in culture using the chemically-defined culture media described herein.

The hPSC populations cultured in the culture media of the disclosure have been demonstrated to maintain the capacity for trilineage differentiation and have been characterized with respect to modulation of marker expression upon cell culture and differentiation (see Example 4).

Various aspects of the invention are described in further detail in the following subsections.

### I. Cells

The starting cells in the cultures are pluripotent stem cells (PSCs), including human pluripotent stem cells (hPSCs). In general, a PSC or hPSC is defined as a stem cell capable of differentiating into all cell types of the adult organism, including those characteristic of each germ cell layer (endoderm, mesoderm, and ectoderm). In an embodiment, an hPSC is an induced pluripotent stem cell (iPSC). In an embodiment, an hPSC is a human embryonic stem cell (hESC), such as from an hESC cell line.

As used herein, the terms “induced pluripotent cell” and “iPSC” refer to a cell taken from a later point in development that has been induced to have expression patterns consistent with a pluripotent cell. The source of the cell can be either embryonic or adult in origin. In an embodiment, the iPSC is the iPSC cell line CR01 (NIH). Additional non-limiting examples of induced pluripotent stem cells (iPSC) include 19-11-1, 19-9-7 or 6-9-9 cells (e.g., as described in Yu, J. et al. (2009) Science 324:797-801).

The terms “human embryonic stem cell and “hESC”, as used herein, refer to pluripotent cells derived from the inner cell mass of human blastocyst embryos. The inner cell mass refers to a mass of cells positioned within the anterior region of the early blastula that gives rise to the entire embryo proper. Non-limiting examples of human embryonic stem cell lines include ES03 cells (WiCell Research Institute) and H9 cells (Thomson, J.A. et al. (1998) Science 282:1145-1147).

Human pluripotent stem cells (PSCs) express cellular markers that can be used to identify cells as being PSCs. Non-limiting examples of pluripotent stem cell markers include SOX2, OCT4, NANOG, TRA-1-60, SSEA4, TRA-1-81, TRA-2-54, SSEA1, SSEA3, SSEA4, CD9, CD24 and OCT3.

In certain embodiments, upon culture of hPSCs in the culture media of the disclosure comprising HS and PEG, following by challenging the cells for ecto-, endo-or meso-dermal activity increases expression of one or more ecto-, endo-or meso-dermal markers, respectively. Challenge of the cells for lineage-specific activity can comprise, for example, stimulating the cells to differentiate along one of indicated lineages. In an embodiment, ectodermal markers whose expression can be stimulated on the cells are PAX6 and/or Nestin. In an embodiment, endodermal markers whose expression can be stimulated on the cells are FOXA2 and/or SOX17. In an embodiment, mesodermal markers whose expression can be stimulated on the cells are TNNT2 and/or Brachyury (T).

In an embodiment, culture of the hPSCs in a culture media comprising HS and PEG leads to downregulation of expression of certain genes. In an embodiment, expression of TGFβ family genes, such as BMP2 and/or BMP4, is downregulated. In an embodiment, expression of Wnt family genes, such as Wnt4, FRZB, FZD5 and/or FRZ8, are downregulated. In an embodiment, expression of retinoic acid family genes, such as CYP26A1, CYP26C1, DHRS3 and/or

CRABP2, are downregulated. In an embodiment, ectodermal genes, such as SOX1, OLIG3, LHX5 and/or OTX2, are downregulated.

### II. Culture Media Components

The method of the disclosure for maintaining hPSC aggregate stability and pluripotency comprise culturing hPSCs in a culture media comprising specific chemical compounds.

In an embodiment, the disclosure provides a culture media for maintaining aggregate stability and pluripotency by culturing hPSCs in a culture media comprising heparin sodium salt (HS) and polyethylene glycol (PEG).

Heparin sodium salt (HS), commercially available in the art (CAS Number 9041 Aug. 1), is the sodium salt form of heparin, which produces an anticoagulant effect by activating antithrombin. In an embodiment, HS is present in the media at a concentration range of 0.01-1.0ug/ml, 0.05-0.5 ug/ml, 0.05-0.2 ug/ml or 0.03-0.15 ug/ml. In an embodiment, HS is present in the media at a concentration of 0.1 ug/ml.

Polyethylene glycol (PEG) is a polyether commercially available in the art (CAS Number 25322-68-3). Its structure is commonly expressed as H—(O—CH₂—CH₂)ₙ—OH. In an embodiment, PEG is present in the media at a concentration range of 2-20 mg/ml, 5-15 mg/ml, 7.5-12.5 mg/ml or 9-11 mg/ml. In an embodiment, PEG is present in the media at a concentration of 10 mg/ml.

In other embodiments, the concentration of PEG in the media is expressed as a weight percentage. For example, in embodiments, PEG is present in the culture media at a concentration of 0.5%-2%. In various embodiments, PEG is present in the culture media at a concentration of 0.5% or 1% or 2%.

In embodiments, the molecular weight (MW) of PEG used in the media can be in a range of 1500-8000 Daltons. In an embodiment, the MW of PEG used in the media is 1500 Daltons. In another embodiment the MW of PEG used in the media is 8000 Daltons.

In an embodiment, the culture media further comprises polyvinyl alcohol (PVA). PVA is a water-soluble polymer commercially available in the art (CAS Number 9002-89-5). Its structure is commonly expressed as [CH2CH (OH)]ₙ. In an embodiment, PVA is present in the media at a concentration range of 0.2-2 mg/ml, 0.5-1.5 mg/ml, 0.75-1.25 mg/ml or 0.9-1.1 mg/ml. In an embodiment, PVA is present in the media at a concentration of 1 mg/ml.

In an embodiment, the culture media further comprises dextran sulfate (DS). DS is a commercially available polyanionic polymer (CAS Number 9011-18-1). It is a highly branched polysaccharide with 1,6 and 1,4 glycosidic linkages. In an embodiment, DS is present in the media at a concentration range of 10-1000 ug/ml, 50-200 ug/ml, 30-150 ug/ml or 90-110 ug/ml. In an embodiment, DS is present in the media at a concentration of 100 ug/ml.

In an embodiment, the culture media further comprises Pluronic acid F-68 (PA). PA is a commercially available non-ionic surfactant (CAS Number 9003 Nov. 6). In an embodiment, PA is present in the media at a concentration range of 0.2-2 mg/ml, 0.5-1.5 mg/ml, 0.75-1.25 mg/ml or 0.9-1.1 mg/ml. In an embodiment, PA is present in the media at a concentration of 1 mg/ml.

### III. Culture Conditions

In combination with the chemically-defined and optimized culture media described in subsection II above, the methods of maintaining hPSC aggregate stability and pluripotency of the disclosure utilize standard culture conditions established in the art for cell culture. For example, cells can be cultured at 37° C. and under 5% CO₂ conditions. A basal media typically is used as the starting media to which supplemental agents are added. Suitable basal media are commercially available in the art. Non-limiting examples of suitable basal media include Essential 8 (E8), DMEM, IMDM and RPMI media. In an embodiment, the basal media is Essential 8.

Pluripotency of the hPSCs in culture can be assessed by evaluating expression of one or more pluripotent stem cell biomarkers. For example, stem cell marker expression can be evaluated over time in culture by standard methods, such as flow cytometry or RNAseq analysis. Non-limiting examples of suitable stem cell markers for assessing pluripotency include SOX2, OCT4, NANOG, TRA-1-60 and SSEA4. In an embodiment, the stem cell marker is SOX2.

Proliferation of the hPSCs in culture can be assessed by evaluating expression of one or more proliferation biomarkers. For example, proliferation marker expression can be evaluated over time in culture by standard methods, such as flow cytometry or RNAseq analysis. Non-limiting examples of suitable markers for assessing hPSC proliferation include MIK67, PCNA and TP53.

In various embodiments, the hPSCs express at least one, at least two, at least three or at least four stem cell-associated biomarkers or proliferation biomarkers. In an embodiment, cells are cultured for sufficient time to increase the expression level of at least one stem cell-associated biomarker or proliferation biomarker, for example by at least 5%, 10%, 15%, 20%, 25% or 50% as compared to the starting cell population. The level of expression of genetic markers in the cultured cells can be measured by techniques available in the art (e.g., RNAseq analysis).

In an embodiment, cells are cultured in the culture media comprising HS and PEG for at least 24 hours, or at least 48 hours, or at least 72 hours or at least 96 hours or at least 1, 2, 3, 4, 5, 6 or 7 days or at least one week or more.

The culture media typically is changed regularly to fresh media. For example, in various embodiments, media is changed every 24, 48 or 72 hours.

### IV. Uses

The methods and compositions of the disclosure for maintaining aggregate stability and pluripotency of human pluripotent stem cells (hPSCs) allow for efficient and robust culture of hPSCs in a variety of culture contexts and for a variety of uses. In an embodiment, the methods and compositions are used to culture hPSCs in bioreactors. In an embodiment, the methods and compositions are used to expand hPSCs to obtain cells to study pluripotent stem cell biology. In an embodiment, the methods and compositions are used to expand hPSCs to obtain cells for further differentiation into more mature cell types, e.g., progenitor cells of a desired cellular lineage. In an embodiment, the methods and compositions are used to expand hPSCs to obtain cells for regenerative medicine purposes. Accordingly, in embodiments, the methods and compositions of the disclosure can be used in connection with the treatment of various diseases and disorders that can benefit from stem cell-derived therapies.

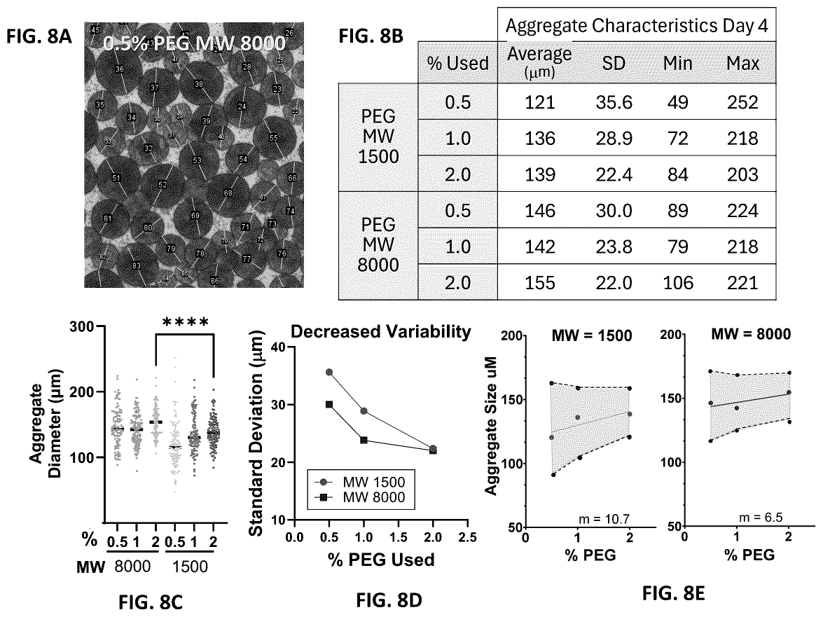

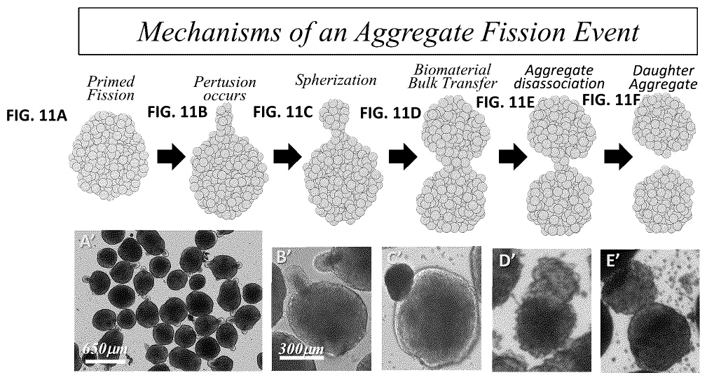

Furthermore, as described in Example 5, varying the concentration of components in the culture media, in particular the concentration and/or molecular weight (MW) of PEG, has been demonstrated to modulate the size and/or stability of hPSC aggregates. For example, as shown in FIGS. 8A-8E, use of PEG with a MW of 1500 Daltons in the culture media led to aggregates of a smaller size than use of PEG with a MW of 8000 Daltons, with the size of the aggregates increasing as the % concentration of PEG in the media increased, regardless of the MW of PEG used. Moreover, hPSCs in culture were shown to maintain their proliferative capacity while not exhibiting significant contact inhibition, and to exhibit daughter aggregate formation. While not intending to be limited by mechanism, FIGS. 11A-11F provides a schematic illustration of an aggregate fission event leading to the formation of daughter aggregates.

Accordingly, in another aspect, the disclosure pertains to a method of modulating size and/or stability of human pluripotent stem cell aggregates in culture using the chemically-defined culture media disclosed herein. In embodiments, the method comprises:

culturing human pluripotent stem cells (hPSCs) in a culture media comprising heparin sodium salt (HS) and polyethylene glycol (PEG), wherein HS and PEG are at a concentration and molecular weight sufficient to modulate the size or stability of hPSCs aggregates in culture.

In embodiments, a concentration and/or molecular weight of PEG is selected that destabilizes larger aggregates. In embodiments, a concentration and/or molecular weight of PEG is selected that mediates creation of smaller aggregates from larger aggregates. In embodiments, a concentration and/or molecular weight of PEG is selected that initiates formation of daughter aggregates as a function of aggregate size or growth.

As used herein, the term “smaller aggregates” is intended to refer to cell aggregates that on average are less than 140 um in size, whereas “larger aggregates” is intended to refer to cell aggregates that on average are equal to or greater than 140 um in size. As used herein, the term “daughter aggregate” is intended to refer to a cell aggregate that originates from another aggregate, such as by a fission event (e.g., as illustrated schematically in FIGS. 11A-11F).

In embodiments, the concentration of PEG in the culture media is varied to select a concentration sufficient to modulate the size or stability of the hPSCs aggregates in culture. In other embodiments, the molecular weight of PEG in the culture media is varied to select a molecular weight sufficient to modulate the size or stability of the hPSCs aggregates in culture. In yet other embodiments, both the concentration and the molecular weight of PEG in the culture media are modulated to select a concentration and molecular weight sufficient to modulate the size or stability of the hPSCs aggregates in culture.

In embodiments, the concentration of PEG in the culture media is in a range of 0.5%-2%. In embodiments, the concentration of PEG in the culture media is 0.5%. In embodiments, the concentration of PEG in the culture media is 1%. In embodiments, the concentration of PEG in the culture media is 2%. In embodiments, the molecular weight of PEG in the culture media is in a range of 1500-8000 Daltons. In embodiments, the molecular weight of PEG in the culture media is 1500 Daltons. In embodiments, the molecular weight of PEG in the culture media is 8000 Daltons. In addition to HS and PEG, the culture media can comprise other media components as described herein.

### V. Compositions

In other aspects, the disclosure provides compositions related to the methods of maintaining hPSC aggregate stability and pluripotency, including culture media and isolated cell cultures.

In one aspect, the disclosure provides an aqueous culture media for maintaining aggregate stability and pluripotency of human pluripotent stem cells (hPSCs) comprising heparin sodium salt (HS) and polyethylene glycol (PEG). In an embodiment, the culture media further comprises polyvinyl alcohol (PVA). In an embodiment, the culture media further comprises dextran sulfate (DS). In an embodiment, the culture media further comprises Pluronic acid F68 (PA). Non-limiting examples of concentration ranges and concentrations for the media components include those described in subsection II above. For example, in an embodiment, the culture media comprises HS at a concentration within a range of 0.05-0.2 ug/ml, e.g., at a concentration of 0.1 ug/ml. In an embodiment, the culture media comprises PEG at a concentration within a range of 5-20 mg/ml, e.g., at a concentration of 10 mg/ml.

In other embodiments, the culture media comprises PEG at a concentration in a range of 0.5%-2%, or at a concentration of 0.5%, or at a concentration of 1% or at a concentration of 2%.

In other embodiments, the culture media comprises PEG at a molecular weight (MW) in a range of 1500-8000 Daltons, or at a MW of 1500 Daltons or at a MW of 8000 Daltons.

In another aspect, the disclosure provides an isolated cell culture of human pluripotent stem cells, the culture comprising human pluripotent stem cells (hPSCs) cultured in a culture media comprising heparin sodium salt (HS) and polyethylene glycol (PEG). In an embodiment, the hiPSCs are human induced pluripotent stem cells (hiPSCs). In an embodiment, the hPSCs are human embryonic stem cells. In an embodiment, the culture media further comprises polyvinyl alcohol (PVA). In an embodiment, the culture media further comprises dextran sulfate (DS). In an embodiment, the culture media further comprises Pluronic acid F68 (PA). Non-limiting examples of concentration ranges and concentrations for the media components include those described in subsection II above. For example, in an embodiment, the culture media comprises HS at a concentration within a range of 0.05-0.2 ug/ml, e.g., at a concentration of 0.1 ug/ml. In an embodiment, the culture media comprises PEG at a concentration within a range of 5-20 mg/ml, e.g., at a concentration of 10 mg/ml.

The present invention is further illustrated by the following examples, which should not be construed as further limiting. The contents of figures and all references, patents and published patent applications cited throughout this application are expressly incorporated herein by reference.

### EXAMPLES

### Example 1: Materials and Methodology

This example describes materials and methods used in the subsequence examples.

### Generating DoE Designs and Perturbation Matrixes

The DoE runs were computer generated from MODDE software (Sartorius Stedim Data Analytical Solutions, SSDAS) using D-optimal interaction designs. All components tested and outputs measured were manually inputted into Design Wizard in MODDE. Factors known to impact the aggregate stability in suspension culture were chosen for the design. Reagent concentration ranges were based on previous publications. Design runs were chosen to have 16 reactions conditions within the addition of 3 center point conditions. One of the reactions had no additives, which is the E8 bioreactor. The highest G-efficiency design was chosen from the DoE designs generated by the software. Media reactions were made manually and filtered before use.

### Human Induced Pluripotent Stem Cell Culture

All hiPSCs were maintained at 37° C. and 5% CO2. E8 medium was purchased ready in solution. Other medium components were purchased as powder. Prior to the assay, cells were grown to 70-80% confluence after 4 days of culture on vitronectin coated 6 well plates. Cells were dissociated with TrypLE, for 3 minutes at 37° C. and resuspended in E8 medium, transferred to 50 ml conical tubes, and centrifuged at 400×g for 6 minutes. The pellet was resuspended in E8 and Y-27632 ROCK inhibitor. 11 million cells were seeded in a 100 ml bioreactor with the corresponding mediums.

### Cell Counting and Aggregate Size

Samples from the bioreactor were taken after slightly mixing using a pipette to avoid sampling bias and gradient formation after settling. 3 ml total was sampled daily for 3 samples of 1 ml for cell count. The cells were first dissociated using Accutase and incubated for 10minutes. Cells were then quenched with E8 then centrifuged at 400×g for 6 minutes. The pellet was resuspended in the same volume of PBS. Samples were then analyzed for total cell count using Attune flow. 3 replicate counts were collected for each bioreactor for each day of culture. 2 samples were collected for aggregate imaging of 500 ul and put on a 24 well plate. EVOS 7000 was used for bright field images of the aggregates. ImageJ was then used to analyze aggregate size and distribution. A minimum of 30 aggregates were measured for each bioreactor every day of the culture. This data was then analyzed for standard deviation on excel and growth rate using GraphPad Prism9. The cell counts were also analyzed for doubling time using GraphPad Prism. Average data from aggregate size and counts were then inputted into the response tab in MODDE. Using the aggregate size generated from day 1 from all bioreactors and their corresponding cell growth rate, a predicted aggregate size was calculated for day 3 and compared to the actual size measured. The predicted aggregate size was calculated from theoretical aggregate volume. Assumptions made were the size of the aggregate diameter being 10 μm based on in house measurements and 64% random aggregate packing density based on empirical observations and simulation experiments (Wilken et al. (2021) Physical Rev. Letters 127:038002).

Aggregate size measured on Day1 was used to calculate aggregate volume using equation:

$${V}_{1} = ( 4 / 3 ) \cdot π \cdot {R}^{3} \qquad ( 1 )$$

The aggregate volume was divided by the individual cell volume defined as

$${V}_{c} = ( 4 / 3 ) \cdot π \cdot {( 10 / 2 )}^{3 =} 523.6 \qquad ( 2 )$$

The division was multiplied by the packing density to obtain cell number in an aggregate volume:

$${N}_{1} = V 1 / {V}_{c} . 0.64 \qquad ( 3 )$$

The predicted cell number of an aggregate after (3) days in culture was calculated from the cell growth rate measured from daily count (K) using the equation below:

$${N}_{3 P} = {N}_{1} * exp ( K * 3 ) \qquad ( 4 )$$

From the cell number, the predicted aggregate volume (V_{3P}) was calculated again from the first equation. We defined the difference between the predicted and actual size as “Aggregate % Error”. This is calculated by subtracting the predicted from the actual average size measured and divided by the actual value:

$$Aggregate \% Error = ( {V}_{3} - {V}_{3 P} ) / {V}_{3} \qquad ( 5 )$$

Flow Cytometry and qPCR Testing

3 ml sample was taken from each bioreactor on day 4 of the culture for qPCR testing. RNA samples were dissolved in Trizol and extracted. Quantification of RNA was performed on epoch reader. A high-Capacity cDNA RT Kit was used for reverse transcription of RNA samples of each bioreactor. 3 replicate samples of cDNA were obtained from 3 replicate samples of each bioreactor. Data collection was performed using QuantStudio for qPCR testing. The runs were performed per the manufacturer's protocol and recommendations. The primers used were NANOG, SOX2, POU5F1 and GAPDH. The data set obtained was then exported to Excel and normalized against corresponding housekeeping gene GAPDH. Final expression levels were expressed as 1/(2^{Crt})×1000 and inputted into the MODDE software. A 3 ml sample was taken from each bioreactor on day 4 of the culture for flow testing. The aggregates were dissociated with Accutase for 10 minutes into single cells. Cells were resuspended in PBS and divided into samples for intracellular staining and cells for extracellular staining. Sample for intracellular staining were fixed with a live/dead stain FVS 780, then permeabilized and stained for OCT4 and SOX2.

### Statistical Methods

Data were analyzed and graphed in Excel, R, GraphPad Prism9 and MODDE software. Modeling of design space was performed using MODDE software. Comparisons were conducted via ANOVA test with a significant difference defined as P<0.05. The sample size of bioreactor run was predetermined by the MODDE generator. The experiments were not randomized for blocks before testing.

### Example 2: Use of a Design of Experiment Approach to Evaluate Pluripotent Aggregate Expansion

This example utilizes a method of High-Dimensional Design of Experiments (HD-DoE), as previously described in Bukys et al. (2020) Iscience 23:101346. The method employs computerized design geometries to simultaneously test multiple process inputs and offers mathematical modeling of a deep effector/response space. The method allows for finding combinatorial signaling inputs that control a complex process, such as during cell culture. It allows testing of multiple plausible critical process parameters, as such parameters impact output responses, such as gene expression. D-optimal DoE interaction designs were used to compress the number of experimental runs compared with a full factorial design. Design compression allows determination interactions and predictions within the design space explored within the concentration range used (i.e., “known space’) (Bukys et al. (2020) iScience 23:101346).

Multiple high-molecular weight polymers have previously been shown to impact pluripotent stem cell cultures, but combinatorial assessments have not been performed to any large extent. Here, we investigated dextran sulfate (DS), Heparin sodium salt (HS), poly (vinyl alcohol) (PVA), Pluronic acid F68 (PA) and polyethylene glycol (PEG), at representative concentrations shown below in Table 1:

TABLE 1

| Components | Concentration | |
|---|---|---|
| PVA | 1 | mg ml⁻¹ |
| DS | 100 | ug ml⁻¹ |
| PA | 1 | mg ml⁻¹ |
| PEG | 10 | mg ml⁻¹ |
| Heparin sodium salt | 0.1 | ug ml⁻¹ |

These factors and their concentration range were chosen based on previous publications about their impact on the iPSC aggregates growth and maintenance. Previous work showed that expansion of hPSCs in stirred-type bioreactors was improved by the addition of Pluronic F68 acting as non-ionic shear protectant (Manstein et al. (2021) Stem Cell Transl. Med. 10:1063-1080). PA has an average molecular weight of 8400 Da and is a triblock copolymer of poly (ethylene oxide)-poly (propylene oxide)-poly (ethylene oxide). PA was also demonstrated to restore cell growth and viability in cell cultures (Hu et al. (2008) Biotechnol. Bioeng. 101:119-127). PVA is a biocompatible synthetic polymer with a molecular weight that ranges between 31,000-50,000. It is commonly used in the pharmaceutical and food industries (Koski et al. (2004) Materials Letters 58:493-497). In a recent study, researchers replaced serum albumin with PVA to develop a culture system that supports long-term expansion of functional mouse hematopoietic stem cells (HSCs) (Wilkinson et al. (2019) Nature 571:117-121). PVA was also evaluated in hiPSC culture to promote proliferation. At 1 mg/mL PVA resulted in maximal cell density within the range tested from 0.1 to 10 mg/ml (Tang et al. (2021) Cell Proliferation 54: e13112). PEG is a polymer based on the —CH2CH2O— repeat unit that comes in different size ranges (Sandier, S. R. and Karo, W. (1980) Polymer Syntheses, Academic Press, New York, Vol. 3, pp. 138-161). Similarly, to PVA, PEG is approved for drug and pharmaceutical applications. It functions as an aggregation inhibitor/emulsifying agent that may improve protein stability (Castellanos et al. (2003) J. Controlled Release 88:135-145). Both heparin sodium salt and DS are polysulphates that may increase aggregate stability and cell growth. Heparin sodium salt supports hiPSC growth due to FGF2 stabilization. FGF2 degrades relatively quickly at 37° C. (Chen et al. (2011) Nature Methods 8:424 429; Furue et al. (2008) Proc. Natl. Acad. Sci. USA 105:13409-13414). Dextran sulphate has been commonly used in the biopharmaceutical industry to prevent cell aggregation. It is a poly-sulphated compound and was recently reported to control aggregate size and shape properties of hiPSCs, without compromising the maintenance of pluripotency (Lipsitz et al. (2018) Biotechnol. Bioeng. 115:2061-2066; Nogueiro et al. (2019) J. Biol. Eng. 13:74).

We generated D-optimal DoE interaction designs to evaluate these effectors. Cultures were maintained for four days in a suspension environment of a 100 ml vertical wheel bioreactor. Reactors were initially seeded from 60-70% confluent hiPSCs pre-cultured on coated vitronectin plates (Braam et al. (2008) Stem Cells 26:2257-2265) that were dissociated into single cells with TrypLE and seeded at 1.1E5 cells/ml in the presence of Rho kinase inhibitor Y-27632 for the first 24 h. Daily aggregate samples were collected to obtain images. On the final day of culture, RNA was extracted and used in cDNA synthesis. Cell samples for flow, counts and plating were digested using Accutase and resuspended in E8+Y-27632 ROCK inhibitor and Pen-Strep processed for FLOW analysis and analyzed with cell state specific antibodies.

Pluripotency data was assessed at the end of the experiment through Immunohistochemistry (IHC), flow cytometry and qPCR testing. The experiment was divided into two batches of experiments to be run at different times. Three replicated center point experiments with all five additives at mid-concentrations were used and split between the batches for normalization and model evaluation purposes. The optimization criteria were based on multiple attributes related to aggregate stability, cell growth and pluripotency. Mathematical models were generated and tuned for maximal predictive power and fit. The model responses for each parameter were statistically evaluated for model reproducibility, model validity, fit (R2) and prediction precision (Q2). All response variables measured had an R2 value above 0.5 indicating a model with high significance. The model had model validity above 0.25 indicating the absence of problems related to outliers and transformation issues.

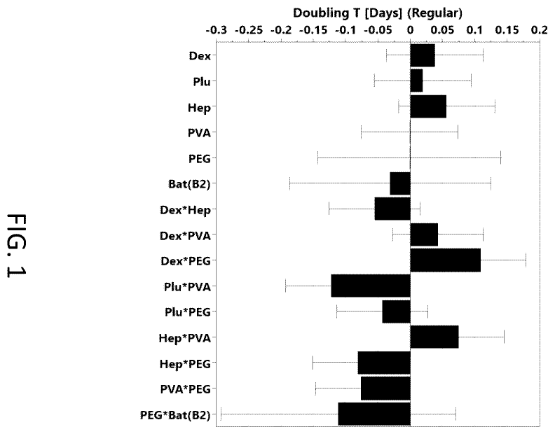

Inspection of the response models revealed insights into the degree of single/combinatorial control of key culture parameters. Invariably, coefficient listings of responses revealed that most significant predictors of culture behavior was contributed by factor interactions, as shown in the representative data shown in FIG. 1.

All bioreactor runs were performed using E8 basal medium for pluripotency maintenance creating a suitable bioreactor growth assay in which the impact of additives upon iPSC cell aggregate stability and growth could be measured. The assay conditions were based on our previous work evaluating process parameters such as bioreactor seeding density, digestion frequency, single cell dissociation, passage time, plate coating, and bioreactor speed. Using the data obtained from the short-term growth assay, prediction models were constructed using computer modeling software. Response parameters included aggregate diameter sizes, cell concentration, growth rates, pluripotency marker expression, cell viability and aggregate variability. Each response model considers individual factor attributes along with interactions between parameters.

### Impact of Additives on Suspension Growth Kinetics

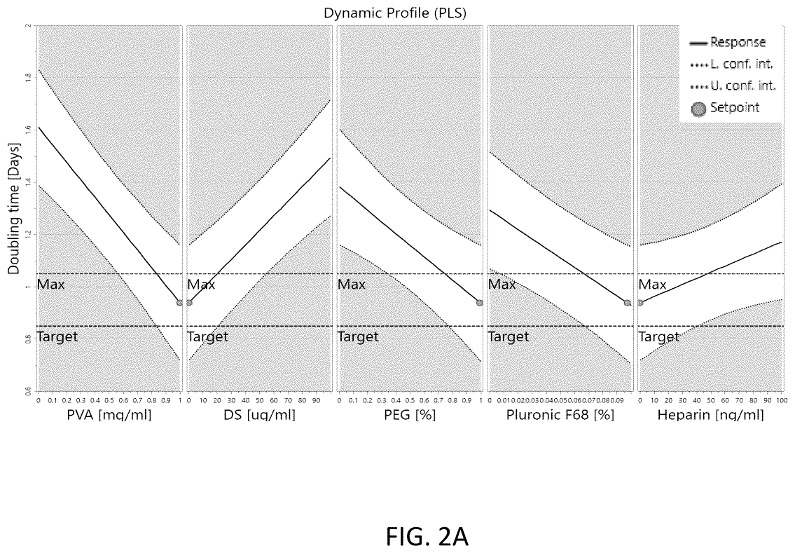

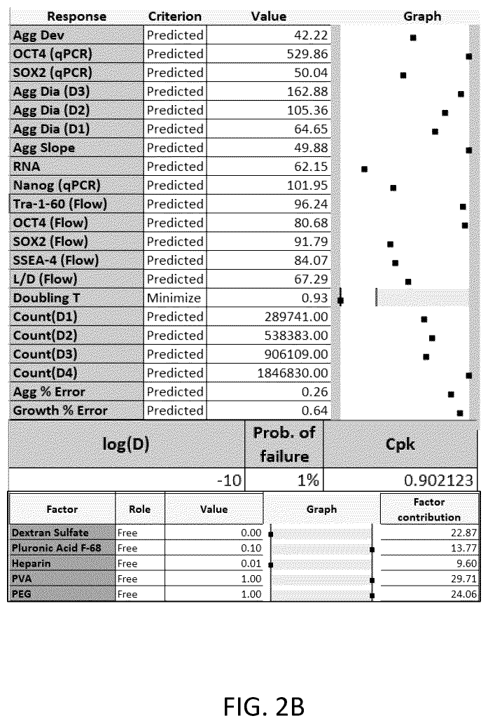

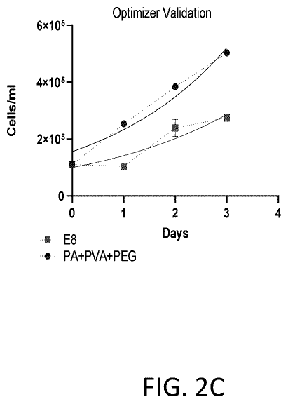

Using doubling time data collected over the 4 days of culture in bioreactors, an optimizer setpoint dynamic profile was performed to detect main factor contributions (FIG. 2A). Desirability criteria were set for the minimization of doubling time (i.e. maximal growth rate, FIG. 2B). Optimizer results detected read out conditions revealed that Pluronic F68, PVA and PEG all contributed to increased proliferation. The process capability index (Cpk) obtained from this optimizer was 0.9 (FIG. 2B) with a probability of failure of 1%. Modeling terms for maximal growth rate control include interaction terms between the additives.

While interpreting and confirming a triple interaction can be complex, the obtained results from the model optimizer and coefficients plot confirm a combined effect of these three additives that is not simply additive through independent factor contributions (FIG. 1) but rather there is a synergistic relationship observed contributing to the desired overall growth in this suspension system.

hiPSC culture without daily media changes presented caveats related to impact of increased cell growth eventually plateauing due to nutrients limitation. Also, production of glycolytic waste products such as lactate and ammonia will reduce the medium pH negatively impacting pluripotency and impair continuous growth. For this purpose, the original seeding density of cells was reduced from the typical 1.5 E5 cells/ml to 1.1 E5 cells/ml and the assay was revalidated at day 3 in addition to day 4 for many variables measured. Validation experiments compared the optimized medium to the control E8 medium without additives (FIG. 2C). The difference between the growth rates of the two mediums confirms the advantage of the additives (k=0.4) over the control (k=0.35).

### Optimization of Pluripotency Maintenance

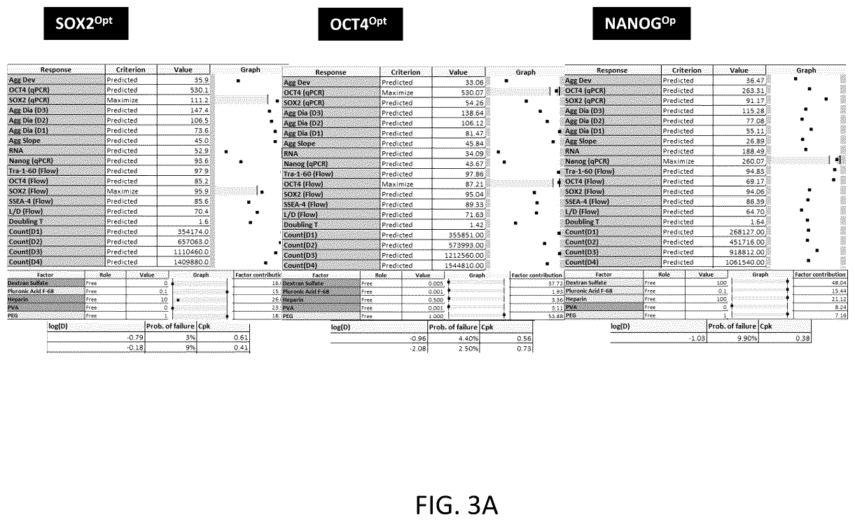

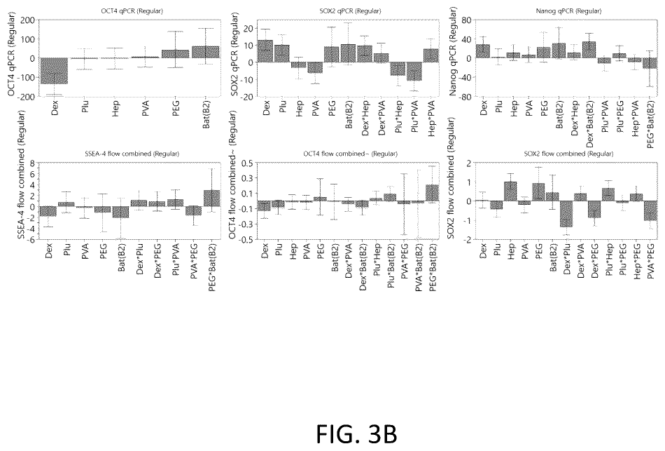

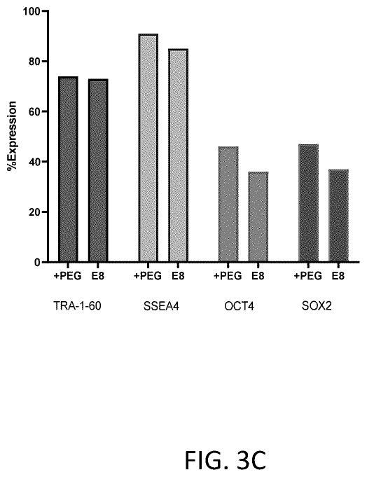

Attaining maximal growth conditions of iPSC is not meaningful unless the iPSC state characteristics is also maintained. To demonstrate the maintenance of pluripotency, it was essential to test for the markers of undifferentiated iPSCs. At the end of the experiment, using flow cytometry we tested for SSEA4, OCT4, SOX2 and TRA-1-60 while a complimentary q-PCR assessment was conducted for NANOG, OCT4 and SOX2. The results of these experiments were used to model the effect of the additives on pluripotency. We separately optimized for the markers individually as shown in FIG. 3A, since model results are non-confounding when optimization is performed for a singular response at a time. This is because that until proven that individual factors are not in regulatory conflict (i.e., SOX2, NANOG, or OCT4 being subject to differential control), single-response factor optimization is prudent. As we found, these factors are indeed subject to differential control.

Model optimizers for SOX2 and OCT4 had PEG as a major contributor with a factor contribution (FC) of 18.72 and 53.88 respectively. The model for SOX2 also contained a significant contribution from Pluronic F68 (FC=15.28 and 3% probability of failure). Optimization of NANOG had a contribution of PEG and Pluronic F68 with FC of 7.16 and 15.44 respectively, however the addition of heparin sodium salt and DS had the highest contribution for NANOG optimization with FC of 21.12 and 48.04 respectively. Expression of pluripotency markers using these optimizers was also validated by immunohistochemistry. qPCR and flow cytometry result data spread across the experimental series demonstrated that OCT4 and NANOG have good response variables as significant differences are needed for generating strong models. In contrast, SOX2, TRA-1-60 and SSEA4 exhibit less variance across the design. Coefficients for all flow and qPCR markers, as shown in FIG. 3B, demonstrated that DS had opposing impact on OCT4 (negative) and NANOG (positive) making it challenging to select additive inputs that would satisfy all pluripotent markers. For the second step validation of the results, the common factor from all optimizers (PEG) was used for determining a model that can be compared to the control media without additives. Validation focused on expression of the pluripotency markers. The results shown in FIG. 3C validated that the addition of PEG improves the expression of pluripotency markers.

Co-expression of pluripotency intracellular markers (SOX2 and OCT4) with the extracellular markers (SSEA4 and TRA-1-60) was validated using flow cytometry analysis. Comparative data showed co-expression of intracellular markers and extracellular co-expression. Models demonstrated a strong correlation of 89% between OCT4 and TRA-1-60 expression. A correlation this strong was not observed between any of the other pluripotency markers. Using model optimizers for the maximization, and minimization, of OCT4 expression confirmed that TRA-1-60 was coregulated with OCT4 expression. This finding shows a direct predictive connection between OCT4 and TRA-1-60 that suggests a coregulatory relationship. The model showed additional correlation between these pluripotency markers and the response variable of aggregate size. Further validation of this correlation was evaluated using two software tools, R and MODDE. A positive correlation of 76-85% was observed between increased diameter size and OCT4/TRA-1-60 expression respectively. Whereas a negative correlation was observed between increased diameter size and SOX2 (-46%) and NANOG (-50%) expression. Correlation results from both statistical tools showed that there is a significant correlation between aggregate size change and the expression of pluripotency markers with a P-value<0.05, therefore the observed correlation is unlikely to occur by chance alone, showing an association between these variables.

### Example 3: Controlling Aggregate Architecture Using Media Additives

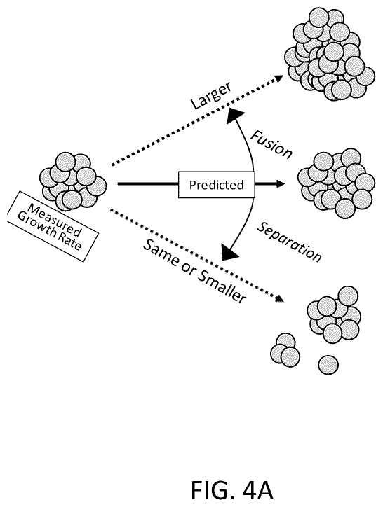

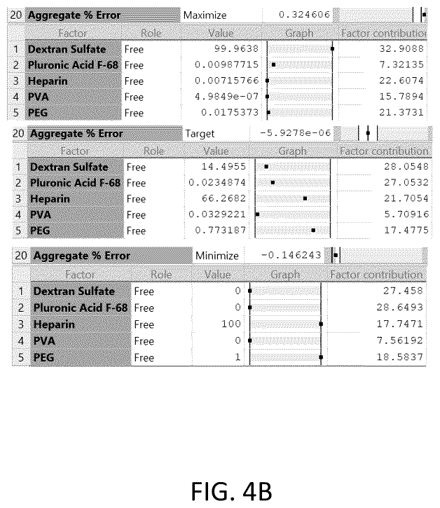

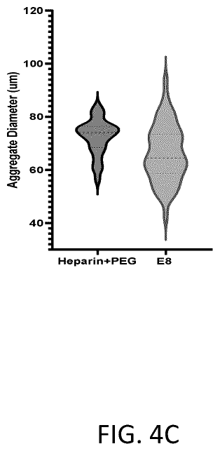

Since aggregate size was demonstrated to impact pluripotency and its heterogeneity could cause inconsistencies in nutrients and oxygen intake by the cells, we sought to establish parameters qualifying the aggregate state. Ideally, optimal aggregate growth should occur through the formation of aggregates from single cells upon seeding, whereafter aggregate size growth should occur due to cell proliferation only. Large deviation in aggregate sizes could have two causes 1) unstable aggregates that break apart due to shear forces and separate to become smaller or 2) aggregate fusion causes larger aggregates to form from the addition of smaller ones (FIG. 4A). Therefore, a parameter can be calculated and set for a desired range in an optimizer (FIG. 4B). Using the data collected on cell proliferation and aggregate size throughout culture time. A violin plot of aggregate diameters in bioreactors on day 3 is shown (FIG. 4C) demonstrating that additives modify the distribution of the observed sizes.

Aggregate sizes measured have a deviation and therefore modelling on average size could potentially mask that response output. To overcome this limitation of using the average aggregate size as a response variable, the average size was inputted in the model in addition to its corresponding standard deviation. Using the average aggregate size generated from day 1 from all reactions in the design with their corresponding growth rates, a predicted aggregate size was calculated for day 3 and compared to the observed size measured. We define this difference as “Aggregate % Error”. This is calculated by subtracting the predicted from the actual average size measured and divided by the observed. Using this derivative assay parameters, it was then possible to obtain a combination of components that would predict stable size limiting, hereby limiting aggregate fusion (maximization) or aggregate instability (minimization) (FIG. 4B). The aggregate diameter deviation was calculated from aggregate diameter measurements of images taken on each culture day using ImageJ software.

A known source of aggregate size increase is the adhesion of two or more aggregates into a large one (Otsuji et al. (2014) Stem Cell Reports 2:746). It is desired that the formation and growth of aggregates is only a contribution of cell proliferation and not fusion between cell spheres. Processes such as changing the media, taking samples, or anything that removes bioreactors from the base, can create static cultures that lead to an environment promoting clump formation. Our optimizer for the target aggregate % error of 0 (FIG. 4B), revealed that the main contributors to decreasing the aggregate size variance in culture were low concentrations of DS and pluronic F68 with a contribution factor of 28.05 and 27.05 respectively, whereas presence of heparin sodium salt (21.71) and PEG (17.48) contributed to stabilizing aggregate size variance.

Inspection of the aggregate diameters and growth slope showed a decrease in this optimization which is desirable as it indicates control over aggregation growth. The cell state control was also evaluated at this setpoint. NANOG expression was close to maximum indicating pluripotency maintenance upon aggregate control. When looking at a comparison of the aggregate % error within the bioreactors, the bioreactor with the minimum error and deviation value was reaction 15 which was composed of a media containing DS, PVA, PEG and heparin sodium salt. Validation comparing the optimizer results to the control with the E8 medium with no additives (FIG. 4C) shows that the aggregate size spread was minimized with the addition of heparin sodium salt and PEG.

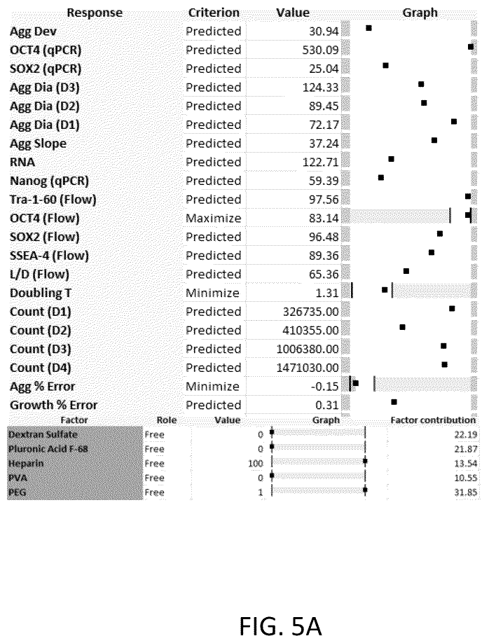

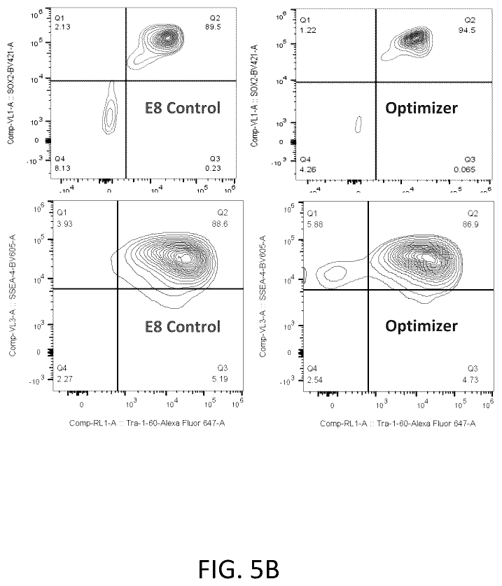

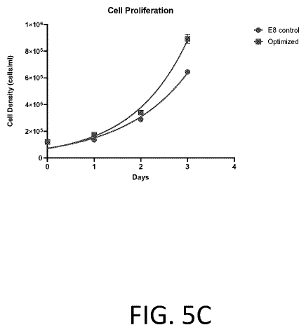

Using an optimizer model, we determined the combination of factors that satisfies an overall desired output of growth rate, pluripotency, and aggregate stability (FIG. 5A), not sacrificing one over another. The results suggested that the combination of heparin sodium salt and PEG can be added to maintain aggregate stability, pluripotency, and growth aspects simultaneously against an E8 control and maintenance of pluripotency (FIG. 5B), aggregate stability and cell proliferation (FIG. 5C) were specifically addressed. Results validated the robustness and repeatability of the predicted model. The Aggregate stability parameter showed that its optimization can indirectly provide pluripotency maintenance and growth throughout the assay. Therefore, this parameter can be regarded as a critical attribute that has a significant impact on iPSC culture in a suspension environment.

### Example 4: Characterization of Pluripotent Stem Cells Cultured in Media Comprising PEG and HS

In this example, various characteristics of the cultured pluripotent stem cells (e.g., iPSC aggregates) were evaluated, including their differentiation potential and karyotyping stability.

The following methodologies were used:

### Tri-lineage Differentiation

iPSCs that were maintained and cultured in the medium described in Example 2 were cryopreserved in 10% DMSO and recovered on 6-well plates to be tested for differentiation potential. The cells were differentiated using the STEMdiff™ Trilineage Differentiation Kit protocol (STEMCELL Technologies). Immunofluorescence was carried out using antibodies for endoderm, ectoderm, and mesoderm lineage specific markers.

### Karyotyping Analysis

Karyotyping was characterized by WiCell Research Institute on live cells that were maintained in optimized medium. The results showed normal karyotype with no clonal abnormalities detected at the stated band level of resolution (425-450).

### RNA Isolation, Library Preparation and RNA-seq

The RNA was qualified by using Qubit™M RNA BR Assay Kit (ThermoFisher-Catalogue No-Q10211). Total RNA was isolated using MagMAX™-96 Total RNA Isolation Kit

(ThermoFisher Scientific) according to the manufacturer's instructions. RNA quality was validated using 4200 TapeStation System (Agilent Technologies). Enrichment of polyadenylated RNA and library preparation were performed using Illumina Stranded mRNA Prep (illumina) using the reagents provided in an Illumina® TruSeq® Stranded mRNA library prep workflow. The library underwent a final cleanup using the Agencourt AMPure XP system (Beckman Coulter) after which the libraries' quality was assessed using a 4200 TapeStation System (Agilent Technologies).

For all samples, the sequencing was done at Genewiz from Azenta Life Sciences. The quality trimming and alignment of the samples were conducted using the nextflow nf-core/rnaseq pipeline (version 3.6). The pipeline incorporated Trim Galore (v.0.6.7) for adaptor trimming and quality control. The trimmed RNAseq reads were then mapped to the Homo sapiens GRCh38 genome annotation utilizing STAR (v 2.6.1). Datasets underwent filtration to eliminate low counts (<10 reads), and differentially expressed genes were identified using the “DESeq2” (v 1.40.2) package in “R” (4.3.1). The heatmaps was created using the package “pheatmap” (v1.0.12).

### Results

To further evaluate the pluripotent nature of the sized controlled aggregates, their tri-lineage differentiation potential and karyotyping stability were evaluated. The ability for trilineage differentiation was sustained in aggregates, as evident through the selective activation of PAX6 and NES, the activation of FOXA2 and SOX17, and the activation of T and TNNT2 when challenged for ectodermal, endodermal, and mesodermal differentiation respectively.

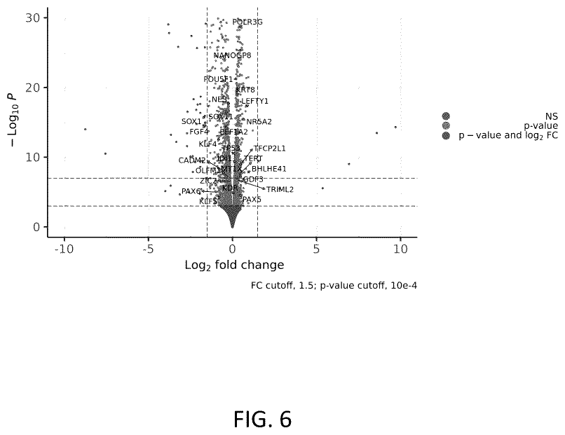

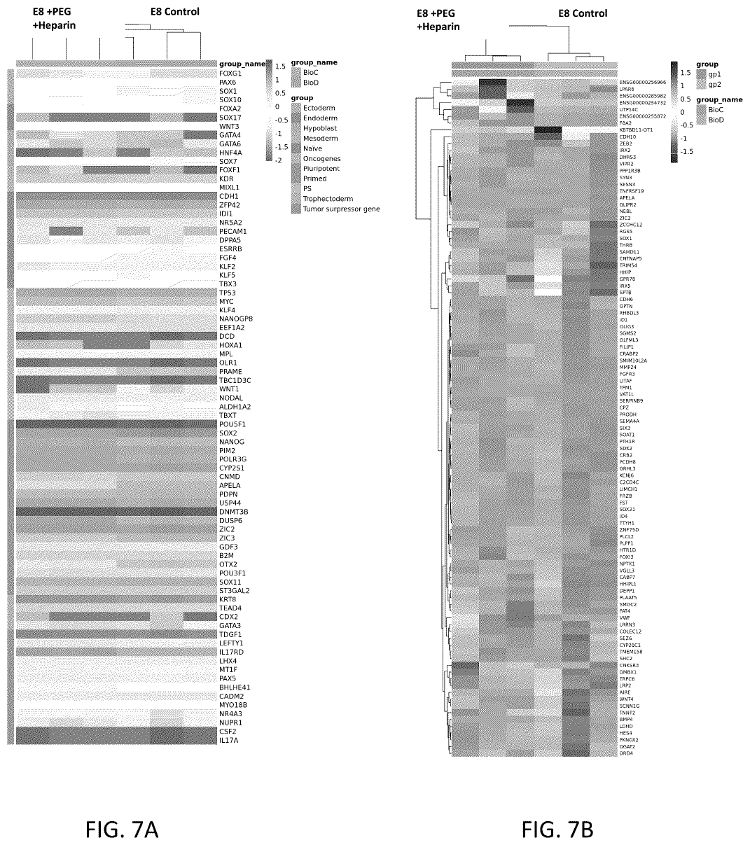

This pluripotent assessment was complimented through RNA sequencing, the results of which are shown in FIG. 6 and FIGS. 7A-7B. Volcano plots comparing the samples showed that few genes were responsive to the addition of PEG and HS (FIG. 6). Evaluation of core pluripotent genes showed that expression levels were similar between samples with a clear indication of a primed phenotype (FIG. 7A). In addition, several well-known oncogenes were evaluated, notably TP53, MYC, NANOGP8, EEF1A2 and KLF4, which were expressed with similar transcript levels between samples, although KLF4 had low overall expression. Conversely, tumor suppressor genes (TSR) and several early lineage drivers were examined. It was observed that a few genes indicative of forward differentiation were expressed, with TSR genes TDGF1, LEFTY1 and IL17RC and the trophectodermal genes KRT8 and TEAD4 showing comparable expression levels. Expression of the primary primitive streak gene NODAL was observed at low transcript levels in both samples (FIG. 7A).

To gain a better understanding of what transcripts were changing in expression in response to the addition of PEG and HS within bioreactors, a differential expression analysis was performed (FIG. 7B). Examining the top differentially expressed genes we noted that there were several genes involved in ectodermal differentiation and some key signaling pathway components that were significantly downregulated in response to the presence of PEG and HS. Down-regulated ectodermal genes included SOX1, OLIG3, LHX5 and OTX2 as well as some less known ones (FIG. 7B). The pathways most downregulated were the TGFβ family as suggested by BMP2 and BMP4 down regulation, though the previously mentioned levels of TDGF1, LEFTY1 and NODAL remained consistent to cultures lacking additives. Wnt family signaling components down regulated were Wnt4, FRZB, FZD5 and FRZ8. Also a decrease in retinoic acid signaling components were noted, as indicated by decreased CYP26A1, CYP26C1, DHRS3 and CRABP2 transcript levels. These results validated the robustness and repeatability of the culture media and protocol described in Example 2. We conclude that the aggregate stability variable can indirectly provide pluripotency maintenance and growth throughout the assay without impacting cell viability. Therefore, this parameter can be regarded as a critical attribute that has a significant impact on iPSC cultures in a suspension environment.

### Example 5: Mechanistic Evaluation of Pluripotent Stem Cells Grown in the Presence of PEG and HS

To establish how the composition of PEG affects the characteristics of the iPSC aggregates, the molecular weight and percentage of PEG used in bioreactor runs were varied.

The concentration of the HS used throughout this series of experiments was held constant. iPSCs were seeded into media preparations varying these two factors and grown in VWPBS biorcactors for 3 days before analyzing data. FIG. 8A shows a typical result of the analysis performed to measure aggregate size for the reaction that used 0.5% PEG with a molecular weight of 8000. A table showing all the conditions used, average aggregate size generated with the standard deviation and minimum and maximum aggregate size is shown in FIG. 8B. The general trend that was shown through this series of experiments was that as the concentration of the PEG increased the average aggregate size increased (FIG. 8C) while the variability of the aggregate sizes decreased (FIG. 8D and FIG. 8E). As the percentage of PEG used was increased, the size of the aggregates generated becomes more homogenous (FIG. 8D and FIG. 8E) with a lower degree of variability across the entire culture population. These effects were also observed by increasing the molecular weight of the PEG used in the reaction. Increasing the molecular weight resulted in both an increase in aggregate size and less variability in aggregate size across the bioreactor culture as a whole.

All together, these results show that the size of the aggregate generated in a bioreactor can be controlled and influenced through the molecular characteristic of the PEG used. One can determine the optimal aggregate size needed and choose the MW of different PEG formulations and the percentage used to determine the formulation best suited for generating aggregates of a desired size.

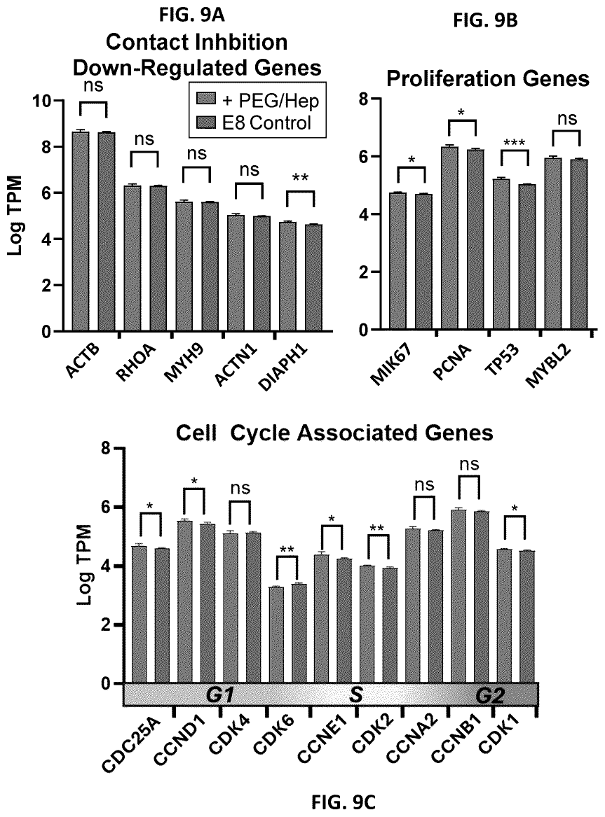

How PEG/HS can control aggregate size was further assessed at the transcript level. One possibility is that PEG is directly interacting with cells and becoming associated at the cellular surface. This would imply that as aggregates grow the continued deposition of PEG could only occur on the outer surface of the aggregate. This would result in the PEG surrounding and covering the growing aggregate, a scenario similar to encapsulation. It is possible that this outer covering layer could begin to limit growth accounting for the overall control of aggregate size. As aggregates grow, they become more constrained imposing a contact inhibition throughout the aggregate which would disallow continuous growth. To address this possibility, genes known to be down-regulated in response to contact inhibition were evaluated (FIG. 9A). Expression levels of the transcripts ACTB, RHOA, MYH9 and ACTN1 showed no significant difference between the two conditions, suggesting no increase in contact inhibition in the iPSCs aggregates generated in the presence of PEG/HS. Only DIAPH1 showed any significant difference in transcript levels and it was down-regulated in the control. All together, these results shows that no significant contact inhibition occurred in the presence of the additives PEG and HS.

In contrast, continued growth within media supplemented with PEG/HS was suggested. An increase in proliferation markers was observed in this condition over the control reaction using only E8 as a growth media. Four general accepted proliferation markers were assayed (FIG. 9B). MIK67, PCNA and TP53 were all significantly up-regulated in the presence of PEG/HS implying increased proliferation. While a fourth proliferation marker, MYBL2, showed no significant difference between the two systems. To confirm this increased proliferative capacity, various markers associated with the different phases of the cell cycle were assayed. Several genes throughout all phases of the cell cycle were shown to be up-regulated in iPSCs grown in the presence of PEG/HS as shown in FIG. 9C.

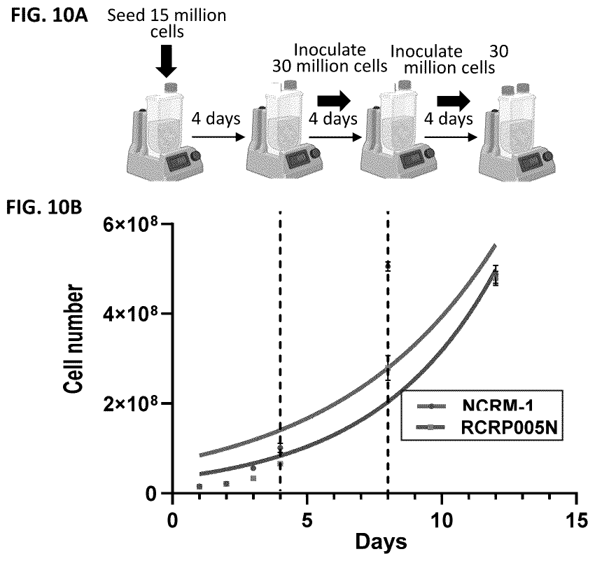

To test the hypothesis that the chemical means of controlling aggregate size was not restricting iPSC growth within a bioreactor, an iPSC culture was taken through a 12-day expansion scenario in which a portion of the bioreactor's biomass was inoculated into a new bioreactor every 4-days (FIG. 10A). It was noted that there was continuous growth throughout this culture phase even in the absence of disrupting the aggregates (FIG. 10B). Altogether this shows that using the additives of PEG/HS has an overall positive effect on iPSC aggregate growth while also limiting and controlling their relative size. The model that would best account for this would suggest that aggregates eventually break apart as would be directly predicted by minimizing for aggregate percent error (FIGS. 4A-4B) from the theoretical aggregate size based on cellular concentration. When this optimizer was originally performed, the intent was to minimize the ability of aggregates from joining together and eliminate fusion events. However, this approach also predicted a destabilized aggregate that would be prone to breaking. What was observe instead is a stabilization of aggregate size throughout bioreactor runs with a minimization of variability of the size ranges within the continuingly growing cultures.

The overall mechanism has been observed and is illustrated schematically in FIGS. 11A-11F and described below.

A) An aggregate is primed for a fission event: Aggregates have been observed to begin to produce pertusion after a media change event (FIG. 11A), suggesting that event can be mediated through a media or temperature change. This may also be triggered when aggregates obtain a larger size, this later occurrence is what can be exploited through modulating the MW and percentage of PEG used in the media.

B) A pertusion occurs: This is observed regularly when performing media changes where cellular material begins darting out from the aggregate (FIG. 11B). This most likely occurs at weak points in PEG/HS covering of the aggregates and results in cellular mass being squeezed out. The growing aggregate in this fashion overcomes the volume constraints and explains why no evidence of contact inhibition was observed within these cultures.

C) A smaller spherical aggregate begins to form: These initiated daughter aggregates are still attached to the parental aggregate (FIG. 11C). Cellular material is transferred into the pertusion, resulting in the formation of a smaller daughter aggregate.

D) Bulk transfer of cellular material occurs: An equalization of the biomass between the two aggregates occurs as the process continues (FIG. 11D).

E) Adjacent aggregates disassociate: The connection between adjacent aggregates weakens and begins to disassociate (FIG. 11E). This may be “pinched off” by the continued deposition PEG/HS.

Separation of similar size aggregates: The process results in an equalization of biomaterial between the daughter aggregates (FIG. 11F).

### Equivalents

Those skilled in the art will recognize, or be able to ascertain using no more than routine experimentation, many equivalents of the specific embodiments of the invention described herein. Such equivalents are intended to be encompassed by the following claims.

1. A method of maintaining aggregate stability of human pluripotent stem cells (hPSCs), the method comprising culturing the human pluripotent stem cells in a culture media comprising heparin sodium salt (HS) and polyethylene glycol (PEG).

2. The method of claim 1, wherein the hPSCs are human induced pluripotent stem cells (hiPSCs).

3. The method of claim 1, wherein the hPSCs are human embryonic stem cells.

4. The method of claim 1, wherein aggregation of the hPSCs is inhibited compared to culture in a media lacking HS and PEG.

5. The method of claim 1, wherein pluripotency of the hPSCs is maintained in culture.

6. The method of claim 1, wherein HS is present in the culture media at a concentration within a range of 0.05-0.2 ug/ml.

7. The method of claim 6, wherein HS is present in the culture media at a concentration of 0.1 ug/ml.

8. The method of claim 1, wherein PEG is present in the culture media at a concentration within a range of 5-20 mg/ml.

9. The method of claim 8, wherein PEG is present in the culture media at a concentration of 10 mg/ml.

10. The method of claim 1, wherein the culture media further comprises polyvinyl alcohol (PVA).

11. The method of claim 1, wherein PVA is present in the culture media at a concentration within a range of 0.5-2 mg/ml.

12. The method of claim 11, wherein PVA is present in the culture media at a concentration of 1 mg/ml.

13. The method of claim 1, wherein the culture media further comprises dextran sulfate (DS).

14. The method of claim 13, wherein DS is present in the culture media at a concentration within a range of 50-150 ug/ml.

15. The method of claim 14, wherein DS is present in the culture media at a concentration of 100 ug/ml.

16. The method of claim 1, wherein the culture media further comprises Pluronic acid F68 (PA).

17. The method of claim 16, wherein PA is present in the culture media at a concentration within a range of 0.5-2 mg/ml.

18. The method of claim 17, wherein PA is present in the culture media at a concentration of 1 mg/ml.

19. The method of claim 1, wherein the culture media comprises a basal media selected from the group consisting of Essential 8, DMEM, IMDM and RPMI.

20. The method of claim 19, wherein the basal media is Essential 8.

21. The method of claim 1, wherein culturing increases expression of at least one pluripotent stem cell marker on the hPSCs.

22. The method of claim 21, wherein the pluripotent stem cell marker is selected from the group consisting of SOX2, OCT4, NANOG, TRA-1-60 and SSEA4.

23. The method of claim 22, wherein the pluripotent stem cell marker is SOX2.

24. A method of maintaining aggregate stability of human pluripotent stem cells (hPSCs), the method comprising culturing the human pluripotent stem cells in a culture media comprising 0.1 ug/ml heparin sodium salt (HS), 10 mg/ml polyethylene glycol (PEG), 1 mg/ml polyvinyl alcohol (PVA) and 100 ug/ml dextran sulfate (DS).

25. An aqueous culture media for maintaining aggregate stability and pluripotency of human pluripotent stem cells (hPSCs) comprising heparin sodium salt (HS) and polyethylene glycol (PEG).

26. The culture media of claim 25, wherein HS is present at a concentration within a range of 0.05-0.2 ug/ml.

27. The culture media of claim 26, wherein HS is present at a concentration of 0.1 ug/ml.

28. The culture media of claim 25, wherein PEG is present at a concentration within a range of 5-20 mg/ml.

29. The culture media of claim 28, wherein PEG is present at a concentration of 10 mg/ml.

30. The culture media of claim 25, which further comprises polyvinyl alcohol (PVA).

31. The culture media of claim 25, which further comprises dextran sulfate (DS).

32. The culture media of claim 25, which further comprises Pluronic acid F68 (PA).

33. An isolated cell culture of human pluripotent stem cells, the culture comprising human pluripotent stem cells (hPSCs) cultured in a culture media comprising heparin sodium salt (HS) and polyethylene glycol (PEG).

34. The isolated cell culture of claim 33, wherein the hPSCs are human induced pluripotent stem cells (hiPSCs).

35. The isolated cell culture of claim 33, wherein the hPSCs are human embryonic stem cells.

36. The isolated cell culture of claim 33, wherein the culture media further comprises polyvinyl alcohol (PVA).

37. The isolated cell culture of claim 33, wherein the culture media further comprises dextran sulfate (DS).

38. The isolated cell culture of claim 33, wherein the culture media further comprises Pluronic acid F68 (PA).

39. A method of modulating size or stability of human pluripotent stem cell aggregates in culture, the method comprising:
culturing human pluripotent stem cells (hPSCs) in a culture media comprising heparin sodium salt (HS) and polyethylene glycol (PEG), wherein HS and PEG are at a concentration and molecular weight sufficient to modulate the size or stability of hPSCs aggregates in culture.

40. The method of claim 39, wherein the concentration and/or molecular weight of PEG is selected to destabilize larger aggregates.

41. The method of claim 39, wherein the concentration and/or molecular weight of PEG is selected to mediate creation of smaller aggregates from larger aggregates.

42. The method of claim 39, wherein the concentration and/or molecular weight of PEG is selected to initiate formation of daughter aggregates as a function of aggregate size or growth.

43. The method of claim 39, wherein the concentration of PEG in the culture media is varied to select a concentration sufficient to modulate the size or stability of the hPSCs aggregates in culture.

44. The method of claim 39, wherein the molecular weight of PEG in the culture media is varied to select a molecular weight sufficient to modulate the size or stability of the hPSCs aggregates in culture.

45. The method of claim 39, wherein the concentration and the molecular weight of PEG in the culture media are varied to select a concentration and molecular weight sufficient to modulate the size or stability of the hPSCs aggregates in culture.

46. The method of claim 39, wherein the concentration of PEG in the culture media is 0.5%.

47. The method of claim 39, wherein the concentration of PEG in the culture media is 1%.

48. The method of claim 39, wherein the concentration of PEG in the culture media is 2%.

49. The method of claim 39, wherein the molecular weight of PEG in the culture media is 1500 Daltons.

50. The method of claim 39, wherein the molecular weight of PEG in the culture media is 8000 Daltons.
